(19) United States
(12) Patent Application Publication
(10) Pub. No.: US 2011/0245285 A1
(43) Pub. Date: Oct. 6, 2011

(54) PROCESS FOR PREPARATION OF ROSUVASTATIN ACETONIDE CALCIUM

(75) Inventors: Pullela Venkata Srinivas, Hyderabad (IN); Sreenivasa Prasad Anegondi, Hyderabad (IN); Shanmughasamy Rajmahendra, Chennai (IN); Thangarasu Ponnusamy, Coimbatore (IN)

(73) Assignee: BIOCON LIMITED, BANGALORE, KARNATAKA (IN)

(21) Appl. No.: 13/121,178

(22) PCT Filed: Oct. 22, 2008
(86) PCT No.: PCT/IN08/00696

§ 371 Date: Apr. 25, 2011

(30) Foreign Application Priority Data
Sep. 9, 2008 (IN) 02187/CHE/2008

(51) Int. Cl. A61K 31/506; C07D 405/14; A61P 3/06

(52) U.S. Cl. 514/275; 544/296

(57) ABSTRACT

The present invention is in relation to a process for preparation of HMG-CoA reductase inhibitor. More particularly, the present invention provides a process for preparation of (3R,5S,6E) 6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid, calcium salt (Rosuvastatin acetonide calcium) which is used for treating hypercholesterolemia.

### FIELD OF INVENTION

The present invention is in relation to a process for preparation of HMG-CoA reductase inhibitor.

### BACKGROUND AND PRIOR ART OF THE INVENTION

The present invention is related to compound and pharmaceutical compositions useful as potent inhibitors of the enzyme 3-hydroxy-3-methylglutaryl-coenzyme A reductase (HMG CoA reductase), pharmaceutical compositions containing such compounds, and a method of inhibiting the biosynthesis of cholesterol employing such pharmaceutical compositions.

Statin drugs are currently the most therapeutically effective drugs available for reducing the level of LDL in the blood stream of a patient at risk for cardiovascular disease. A high level of low density lipoprotein (LDL) in the bloodstream has been linked to the formation of coronary lesions which obstruct the flow of blood and can rupture and promote thrombosis. It is well known that inhibitors of HMG-CoA reductase are effective in lowering the level of blood plasma cholesterol, especially low density lipoprotein cholesterol (LDL-C), in man (cf. M. S. Brown and J. L. Goldstein, New England Journal of Medicine, 305, No. 9, 515-517 (1981). It has now been established that lowering LDL-C levels affords protection from coronary heart disease (cf. Journal of the American Medical Association, 251, No. 3, 351-374 (1984).

### OBJECTIVES OF THE PRESENT INVENTION

The principle objective of the present invention is to provide a process for the preparation of HMG-CoA reductase inhibitor.

Another objective of the present invention is to provide a process for the preparation of (3R,5S,6E) 6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid, calcium salt (Rosuvastatin acetonide calcium).

### STATEMENT OF THE PRESENT INVENTION

Accordingly, the present invention provides a compound of formula I

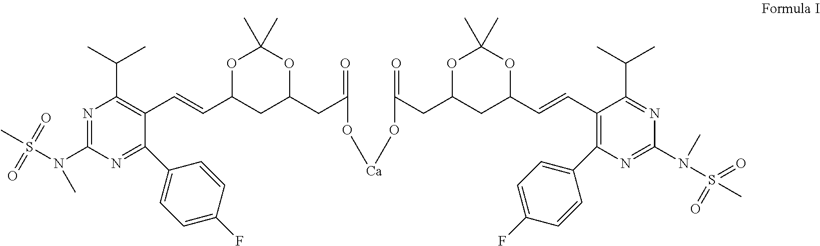

a process for the preparation of compound of formula I of claim 1 comprises, reacting (6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid tert-butyl ester with sodium hydroxide in suitable solvent; and treating the compound obtained from step a with a suitable calcium salt and isolating compound of formula I; and a method of treating hypercholesterolemia comprises compound of formula I in unit dosage form.

### DETAILED DESCRIPTION OF THE PRESENT INVENTION

The present invention is in relation to a compound of formula I

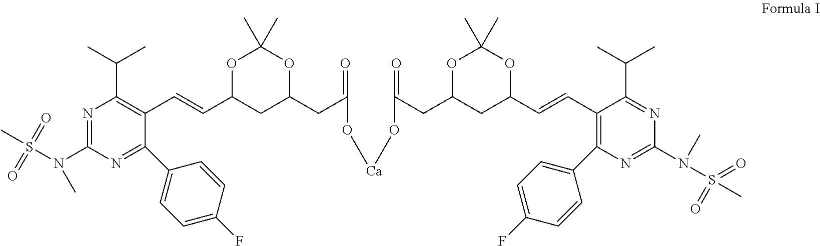

The present invention is in relation to a process for the preparation of compound of formula I of claim 1 comprises, reacting (6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid tert-butyl ester with sodium hydroxide in suitable solvent to obtain a reaction mixture; heating the reaction mixture at a temperature of about 40° C. followed by maintaining the reaction mixture for a time period ranging from about 15 hrs to about 16 hrs; adjusting the pH of the reaction mixture at a temperature of about 30° C. followed by concentration to obtain a compound; and treating the compound obtained with calcium salt followed by isolating the compound of formula I.

In another embodiment of the present invention suitable solvent is selected from the group consisting of ethanol, methanol, isopropyl alcohol, acetonitrile and 1-propanol. In yet another embodiment of the present invention said calcium salt is selecting from the group consisting of calcium acetate, calcium hydroxide and calcium chloride.

The present invention is in relation to a method of treating hypercholesterolemia comprises compound of formula I as claimed in claim 1 in unit dosage form. In accordance with the present invention, it is provided (3R,5S,6E) 6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid, calcium salt (Rosuvastatin acetonide calcium). Which is a potent inhibitor of the enzyme 3-hydroxy-3-methylglutaryl-coenzyme A reductase (HMG CoA reductase). In particular the present invention provides compound of structural formula I.

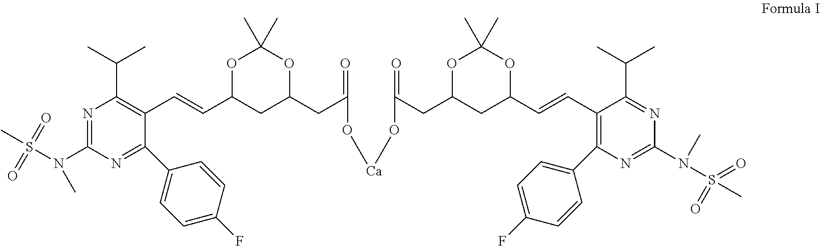

In yet another aspect, the present invention provides pharmaceutical compositions useful as hypolipidemic or hypocholesterolemic agents. The present invention is prepared by hydrolyzing (6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid tert-butyl ester with alkali hydroxide to form a salt further reacted with calcium salt leads to get compound of formula I.

The compound of present invention is (3R,5S,6E) 6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid, calcium salt (Rosuvastatin acetonide calcium) and illustrated in Formula I hereinafter, which compound is an inhibitor of the enzyme 3-hydroxy-3-methylglutaryl-coenzyme A reductase (HMG CoA reductase) and is useful as a pharmaceutical agent, for example in the treatment of hyperlipidemia and hypercholesterolemia as well as other diseases or conditions in which HMG CoA reductase is implicated.

The compound of the invention may be administered to a warm-blooded animal, particularly a human, in need thereof for treatment of a disease in which HMG CoA reductase is implicated, in the form of a conventional pharmaceutical composition. The invention also relates to processes for the preparation of the pharmaceutical compositions.

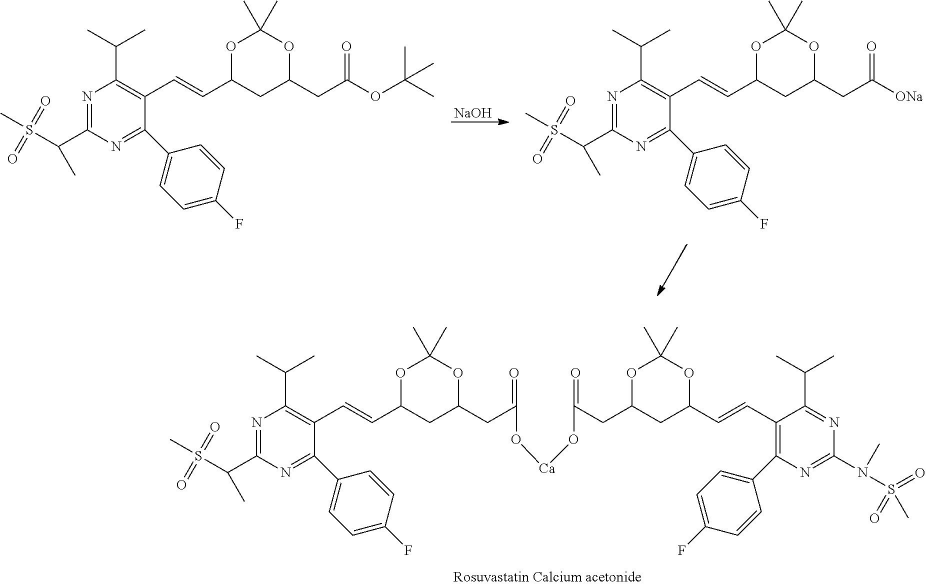

(6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid tert-butyl ester is hydrolyzed with sodium hydroxide followed by treating with calcium salts selected from the group calcium acetate, calcium hydroxide and calcium chloride.

The compound of the present invention can be administered in the form of aqueous or oily suspension, powders, capsules and tablets or parentrally in the form of aqueous or oily suspension or liquid form such as syrup or elixir.

The preparation can be prepared in a conventional manner by using excipients, binders, lubricants, aqueous or oily solubilizers, emulsifier, suspending agents. Preservatives and stabilizers can be further used.

The technology of the instant application is further elaborated with the help of following examples. However, the examples should not be construed to limit the scope of the invention. The following Examples represent preferred embodiments of the present invention.

### Example-1

2.0 g of (6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid tert-butyl ester was taken in 50 mL Ethanol and slowly 4 ml of 50% sodium hydroxide solution was added under stirring. The reaction mixture was heated to 40° C. and maintained at 40° C. for 15 hrs-16 hrs. The pH was adjusted to 9.0-9.5 at 30° C. using 0.5 N HCl. The reaction mass was filtered through celite bed and washed with 4 mL Ethanol. Obtained filtrate was concentrated under vacuum at 50° C. and then stripped of with 5 mL acetonitrile to get a solid. 10 ml of acetonitrile was added to the insolubles and filtered. The filtrates were combined and concentrated. To the concentrated filtrate was added 40 mL Water, 3 mL Methanol & 6 mL MTBE, separated the aqueous layer. Aqueous layer was washed with 6 mL MTBE. Another 6 mL MTBE wash was given to the aqueous layer after adjusting the pH to 8.0-8.2 using 0.5N HCL. The aqueous layer was heated to 40-45° C. and stirred for 30 mins. 0.54 g of Calcium acetate was dissolved in 2.7 ml water followed by slow addition to the reaction mass. The mass was stirred for 30 mins at 40-45° C. and then at 30° C. for 1 h. Solid (3R,5S,6E) 6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid, calcium salt (Rosuvastatin acetonide calcium) obtained was filtered and washed with 6 mL water, dried under vacuum at 45-50° C. for 12 h and packed.

### Example-2

2.0 g of (6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid tert-butyl ester was taken in 50 mL Methanol and slowly 4 ml of 50% sodium hydroxide solution was added under stirring. The reaction mixture was heated to 40° C. and maintained at 40° C. for 15 hrs-16 hrs. The pH was adjusted to pH to 9.0-9.5 at 30° C. using 0.5 N HCl. The reaction mass was filtered through celite bed and washed with 4 mL Methanol. Obtained filtrate was concentrated under vacuum at 50° C. and then stripped of with 5 mL acetonitrile to get a solid. 10 ml of Acetonitrile was added to the insolubles and filtered. The filtrates were combined and concentrated. To the concentrated filtrate was added 40 mL water, 3 mL Methanol & 6 mL MTBE, separated the aqueous layer. Aqueous layer was washed with 6 mL MTBE. Another 6 mL MTBE wash was given to the aqueous layer after adjusting the pH to 8.0-8.2 using 0.5N HCL. The aqueous layer was heated to 40-45° C. and stirred for 30 mins. 0.54 g of calcium acetate was dissolved in 2.7 ml water followed by slow addition to the reaction mass. The mass was stirred for 30 mins at 40-45° C. and then at 30° C. for 1 h. Solid (3R,5S,6E) 6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid, calcium salt (Rosuvastatin acetonide calcium) obtained was filtered and washed with water 6 mL, dried under vacuum at 45-50° C. for 12 h and packed.

### Example-3

2.0 g of (6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid tert-butyl ester was taken in 50 mL Isopropyl alcohol and slowly added 4 ml of 50% sodium hydroxide solution was added under stirring. The reaction mixture was heated to 40° C. and maintained at 40° C. for 15 hrs-16 hrs. The pH was adjusted to 9.0-9.5 at 30° C. using 0.5 NHCL. The reaction mass was filtered through celite bed and washed with 4 mL Isopropyl alcohol. Obtained filtrate was concentrated under vacuum at 50° C. and then stripped of with 5 mL acetonitrile to get a solid. 10 ml of acetonitrile was added to the insolubles and filtered. To the concentrated filtrate was added 40 mL Water, 3 mL Methanol & 6 mL MTBE, separated the aqueous layer. Aqueous layer was washed with 6 mL MTBE. Another 6 mL MTBE wash was given to the aqueous layer after adjusting the pH to 8.0-8.2 using 0.5N HCL. The aqueous layer was heated to 40-45° C. and stirred for 30 mins. 0.54 g of calcium acetate was dissolved in 2.7 ml water followed by slow addition to the reaction mass. The mass was stirred for 30 mins at 40-45° C. and then at 30° C. for 1 h. Solid (3R,5S,6E) 6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid, calcium salt (Rosuvastatin acetonide calcium) obtained was filtered and washed with water 6 mL, dried under vacuum at 45-50° C. for 12 h and packed.

### Example-4

2.0 g of (6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid tert-butyl ester was taken in 50 mL Acetonitrile and slowly 4 ml of 50% sodium hydroxide solution was added under stirring. The reaction mixture was heated to 40° C. and maintained at 40° C. for 15 hrs-16 hrs. The pH was adjusted to 9.0-9.5 at 30° C. using 0.5 N HCL. The reaction mass was filtered through celite bed and washed with 4 mL Acetonitrile. Obtained filtrate was concentrated under vacuum at 50° C. and the stripped of with 5 mL acetonitrile to get a solid. 10 ml of Acetonitrile was added to the insolubles and filtered. The filtrates were combined and concentrated. To the concentrated filtrate was added 40 mL water, 3 mL Methanol & 6 mL MTBE, separated the aqueous layer. Aqueous layer was washed with 6 mL MTBE. Another 6 mL MTBE wash was given to the aqueous layer after adjusting the pH to 8.0-8.2 using 0.5N HCL. The aqueous layer was heated to 40-45° C. and stirred for 30 mins. 0.54 g of calcium acetate was dissolved in 2.7 ml water followed by slow addition to the reaction mass. The mass was stirred for 30 mins at 40-45° C. and then at 30° C. for 1 h. Solid (3R,5S,6E) 6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid, calcium salt (Rosuvastatin acetonide calcium) obtained was filtered and washed with water 6 mL, dried under vacuum at 45-50° C. for 12 h and packed.

### Example-5

2.0 g of (6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid tert-butyl ester was taken in 50 mL 1-Propanol and slowly 4 ml of 50% sodium hydroxide solution was added under stirring. The reaction mixture was heated to 40° C. and maintained at 40° C. for 15 hrs-16 hrs. The pH was adjusted to 9.0-9.5 at 30° C. using 0.5 N HCL. The reaction mass was filtered through celite bed and washed with 4 mL 1-Propanol. Obtained filtrate was concentrated under vacuum at 50° C. and then stripped of with 5 mL acetonitrile to get a solid. 10 ml of Acetonitrile was added to the insolubles and filtered. The filtrates were combined and concentrated. To the concentrated filtrate was added 40 mL water, 3 mL Methanol & 6 mL MTBE, separated the aqueous layer. Aqueous layer was washed with 6 mL MTBE. Another 6 mL MTBE Wash was given to the aqueous layer after adjusting the pH to 8.0-8.2 using 0.5N HCL. The aqueous layer was heated to 40-45° C. and stirred for 30 mins. 0.54 g of calcium acetate was dissolved in 2.7 ml water followed by slow addition to the reaction mass. The mass was stirred for 30 mins at 40-45° C. and then at 30° C. for 1 h. Solid (3R,5S,6E) 6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid, calcium salt (Rosuvastatin acetonide calcium) obtained was filtered and washed with water 6 mL, dried under vacuum at 45-50° C. for 12 h and packed.

### Example-6

2.0 g of (6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid tert-butyl ester was taken in 50 mL Ethanol and slowly 4 ml of 50% sodium hydroxide solution was added under stirring. The reaction mixture was heated to 40° C. and maintained at 40° C. for 15 hrs-16 hrs. The pH was adjusted to 9.0-9.5 at 30° C. using 0.5 N HCL. The reaction mass was filtered through celite bed and washed with 4 mL ethanol. Obtained filtrate was concentrated under vacuum at 50° C. and then stripped of with 5 mL acetonitrile to get a solid. 10 ml of Acetonitrile was added to the insolubles and filtered. The filtrates were combined and concentrated. To the concentrated filtrate was added 40 mL Water, 3 mL Methanol & 6 mL MTBE, separated the aqueous layer. Aqueous layer was washed with 6 mL MTBE. Another 6 mL MTBE Wash was given to the aqueous layer after adjusting the pH to 8.0-8.2 using 0.5N HCL. The aqueous layer was heated to 40-45° C. and stirred for 30 mins. 0.54 g of calcium acetate was dissolved in 2.7 ml water followed by slow addition to the reaction mass. The mass was stirred for 30 mins at 40-45° C. and then at 30° C. for 1 h. Solid (3R,5S,6E) 6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid, calcium salt (Rosuvastatin acetonide calcium) obtained was filtered and washed with water 6 mL, dried under vacuum at 45-50° C. for 12 h and packed.

### Example-7

2.0 g of (6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid tert-butyl ester was taken in 50 mL Ethanol and slowly 4 ml of 50% sodium hydroxide solution was added under stirring. The reaction mixture was heated to 40° C. and maintained at 40° C. for 15 hrs-16 hrs. The pH was adjusted to pH 9.0-9.5 at 30° C. using 0.5 N HCl. The reaction mass was filtered through celite bed and washed with 4 mL Ethanol. Obtained filtrate was concentrated under vacuum at 50° C. and then stripped of with 5 mL acetonitrile to get a solid. 10 ml of Acetonitrile was added to the insolubles and filtered. The filtrates were combined and concentrated. To the concentrated filtrate was added 40 mL water, 3 mL Methanol & 6 mL MTBE, separated the aqueous layer. Aqueous layer was washed with 6 mL MTBE. Another 6 mL MTBE wash was given to the aqueous layer after adjusting the pH to 8.0-8.2 using 0.5N HCL. The aqueous layer was heated to 40-45° C. and stirred for 30 mins. 0.54 g of calcium acetate was dissolved in 2.7 ml water followed by slow addition to the reaction mass. The mass was stirred for 30 mins at 40-45° C. and then at 30° C. for 1 h. Solid (3R,5S,6E) 6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid, calcium salt (Rosuvastatin acetonide calcium) obtained was filtered and washed with water 6 mL dried under vacuum at 45-50° C. for 12 h and packed.

1. A compound of formula I

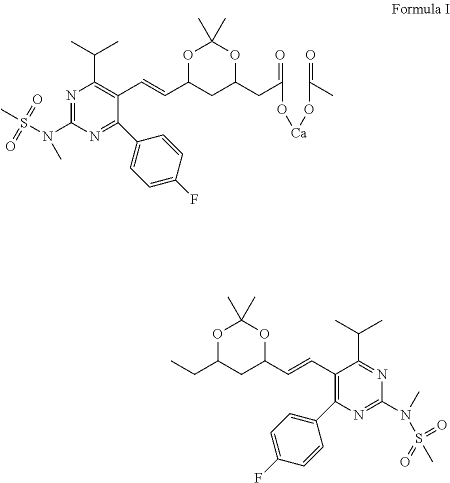

2. A process for the preparation of compound of formula I as claimed in claim 1 said process comprising steps of:
(a) reacting (6-{2-[4-(4-Fluoro-phenyl)-6-isopropyl-2-(methanesulfonyl-methyl-amino)-pyrimidin-5-yl]-vinyl}-2,2-dimethyl-[1,3]dioxan-4-yl)-acetic acid tert-butyl ester with sodium hydroxide in suitable solvent to obtain a reaction mixture;
(b) heating the reaction mixture at a temperature of about 40° C. followed by maintaining the reaction mixture for a time period ranging from about 15 hrs to about 16 hrs;
(c) adjusting pH of the reaction mixture at a temperature of about 30° C. followed by concentration to obtain a compound; and
(d) treating the compound obtained with calcium salt followed by isolating the compound of formula I.

3. The process as claimed in claim 2, wherein suitable solvent is selected from the group consisting of ethanol, methanol, isopropyl alcohol, acetonitrile and 1-propanol.

4. The process as claimed in claim 2, wherein said calcium salt is selected from the group consisting of calcium acetate, calcium hydroxide and calcium chloride.

5. A method of treating hypercholesterolemia comprising administering to a subject in need thereof a compound of formula I as claimed in claim 1 in unit dosage form.

6. (canceled)
